(19) United States
(12) Patent Application Publication
(10) Pub. No.: US 2024/0027428 A1
(43) Pub. Date: Jan. 25, 2024

(54) METHOD FOR BUILDING CO-CULTURE MODEL FOR CACO-2/RAW-264.7 CELLS INDUCED BY LIPOPOLYSACCHARIDES AND APPLICATION OF CO-CULTURE MODEL

(71) Applicant: ZHEJIANG UNIVERSITY OF TECHNOLOGY, Zhejiang (CN)

(72) Inventors: Rongfa Guan, Zhejiang (CN); Mengyu Yang, Zhejiang (CN); Hao Zhong, Zhejiang (CN); Xiaofeng Liu, Zhejiang (CN); Donghong Liu, Zhejiang (CN); Guozhou Cao, Zhejiang (CN); Yong Cheng, Zhejiang (CN); Haitao Shen, Zhejiang (CN); Jun Huang, Zhejiang (CN); Kai Yang, Zhejiang (CN); Yanbin Wang, Zhejiang (CN); Haizhi Huang, Zhejiang (CN)

(73) Assignee: ZHEJIANG UNIVERSITY OF TECHNOLOGY, Zhejiang (CN)

(21) Appl. No.: 18/182,377

(22) Filed: Mar. 13, 2023

(30) Foreign Application Priority Data
Jul. 25, 2022 (CN) 202210878974.4

(51) Int. Cl. G01N 33/50; C12N 5/09

(52) U.S. Cl. CPC G01N 33/5011; C12N 5/0693; C12N 2500/36; C12N 2500/34; C12N 2502/30

(57) ABSTRACT

The present invention discloses a method for building a co-culture model for Caco-2/RAW-264.7 cells induced by lipopolysaccharides and an application of the co-culture model. The method includes: 1) culturing Caco-2 cells and RAW-264.7 cells; 2) inoculating the 5 Caco-2 cells onto an AP side of an upper chamber of a Transwell plate, and incubating; 3) inoculating the RAW-264.7 cells onto a 24-well plate, and incubating; 4) transferring the upper chamber of the Transwell plate into the 24-well plate; 5) dissolving lipopolysaccharides in PBS to prepare a stock solution, filtering, and diluting for later use; and 6) adding an LPS-10% DMEM medium on a BL side, incubating, and forming an intestinal inflammation model.

### CROSS-REFERENCE TO RELATED APPLICATION

This application claims the priority benefit of China application serial no. 202210878974.4, filed on Jul. 25, 2022. The entirety of the above-mentioned patent application is hereby incorporated by reference herein and made a part of this specification.

### BACKGROUND

### Technical Field

The present invention belongs to the technical field of cell culture, and particularly relates to a method for building a co-culture model for caco-2/raw-264.7 cells induced by lipopolysaccharides and an application of the co-culture model.

### Description of Related Art

Cyanidin-3-glucoside (C3G) is the most widely distributed anthocyanin in nature, which is massively present in black rice, black beans, purple cabbages, purple sweet potatoes, black elderberries, blackberries, black grapes, black raspberries, blood oranges, mulberries and other colored grains, fruits, and vegetables. A large number of researches show that C3G has the effects of resisting inflammation, oxidation, and cancer, preventing cardiovascular diseases, and regulating fat metabolism, but the C3G is easily degraded in vivo, most of which in blood circulation are its metabolites. Therefore, it is important to understand the absorption and metabolism of C3G in vivo and the physiological function of metabolites.

Nanoliposomes, after embedding a substance, increases the stability of the embedded substance, and are easily absorbed by cells, and have a slow release effect in vivo. The nanoliposomes are capable of embedding hydrophilic and lipophilic drugs at the same time. Therefore, as an effective substance carrier, the nanoliposomes have a wide application prospect, are often used for embedding special drugs, and may serve as a carrier system for anti-cancer drugs to carry out targeted delivery on the drugs. The drugs reach a tumor site for targeted therapy, which greatly improves the utilization efficiency of the drugs.

Intestinal barrier, one of the most dynamic systems for in vivo metabolism, is crucial to health. The intactness of its epithelial monomolecular layer is of great importance in maintaining the dynamic balance of a host. According to current researches, completely differentiated Caco -2 cells exhibit better morphology and function than other colon cancer cell lines, and form the same cell polarity as small intestinal epithelial cells and dense monolayer tissues. Therefore, Caco-2 cell lines may be used as a good cell model for studying the absorption of drugs. It has been demonstrated by researches that the bioavailability of C3G nanoliposomes in Caco -2 cells is greater than the bioavailability of C3G monomers in Caco-2 cells, and it is indicated that anthocyanin nanoliposomes enter the cells mainly depending on macropinocytosis and clathrin-mediated endocytosis.

“Intestinal leakage” means higher intestinal epithelial permeability, and may cause pathogens, endotoxin, antigens, and proinflammatory cytokines to flow from a gastrointestinal tract into a blood flow and a lymphatic system. As a result, inflammation plays a systematic role in intestinal mucosa. Therefore, a relationship between a permeable intestinal tract and an inflammatory response is bidirectional. It is observed in inflammatory bowel diseases that the destruction of intestinal epithelial barrier is related to the excessive production of oxides, and proinflammatory signals released by intestinal cells and immune cells lead to serious mucosal injury through continuous and aggravated inflammation. Intestinal barrier, one of the most dynamic systems for in vivo metabolism, is crucial to health. The intactness of its epithelial monomolecular layer is of great importance in maintaining the dynamic balance of a host, because it inhibits the invasion of luminal antigens. Therefore, it is necessary to build an in vitro model for cells from an intestinal epithelial cell line Caco-2 located in a top well of a Transwell plate and a macrophage cell line RAW-264.7 located in an outer side well of a substrate, to establish an intestinal inflammatory response for studying a macrophage-mediated absorption mechanism of C3G nanoliposomes by the Caco-2 cells.

### SUMMARY

In view of the problems existing in the prior art, a design objective of the present invention is to provide a method for building a co-culture model for Caco-2/RAW-264.7 cells induced by lipopolysaccharides, which builds a data network of “inflammatory response-absorption law-molecular response”, and illustrates an absorption mechanism of cyanidin-3-glucoside in a co-culture system.

Another objective of the present invention is to provide an application of a co-culture model for Caco-2/RAW-264.7 cells induced by lipopolysaccharides in research on absorption of cyanidin-3-glucoside nanoliposomes by the cells.

Specifically, the present invention is implemented by the following technical solution:

The method for building a co-culture model for Caco-2/RAW-264.7 cells induced by lipopolysaccharides includes the following steps:

1) culturing Caco-2 cells in an MEM medium and RAW-264.7 cells in a DMEM medium under culture conditions of pH 7.4, 5% CO2, 37° C., 95% air, and constant humidity for 21 days, wherein the media are changed once every 1-2 days;

2) inoculating the Caco-2 cells cultured in the step 1) onto an AP side of an upper chamber of a 24-well Transwell plate having a PC membrane of 4μm at a density of 1×10⁵ cells/well, and incubating under the same culture conditions as those in the step 1) for 21 days, wherein a culture medium is changed once every 24 h;

3) inoculating the RAW-264.7 cells onto a 24-well plate at a density of 1×10⁵ cells/well, and incubating under the same culture conditions as those in the step 1) for 1 day;

4) transferring the upper chamber of the Transwell plate loaded with the Caco -2 cells incubated for 21 days to the 24-well plate loaded with the RAW-264.7 cells incubated for 1 day, wherein at this time, the Caco-2 cells are located on the AP side, and the RAW-264.7 cells are located on a BL side;

5) dissolving lipopolysaccharides in PBS to prepare a 50 μg/mL stock solution, filtering through a 0.22 μm pinhole filter, and diluting the stock solution into a 1 μg/mL LPS-10% DMEM medium with a 10% DMEM medium; and

6) adding the 1 μg/mL LPS-10% DMEM medium on the BL side, incubating for 3 h, detecting the concentration of an inflammatory factor on the AP side with an ELISA kit, and forming an intestinal inflammation model.

Further, in the step 1), the MEM medium contains 20% of fetal bovine serum, 1% of HEPES, 1% of NEAA, 1% of L-glutamine, 1% of sodium pyruvate, 100 U/mL of penicillin, and 100 mg/mL of streptomycin.

Further, in the step 1), the DMEM medium contains 10% of fetal bovine serum, 1% of HEPES, 1% of NEAA, 1% of L-glutamine, 100 U/mL of penicillin, and 100 mg/mL of streptomycin.

Further, in the step 2), a transepithelial electrical resistance of the cells is detected once every three days on average, and the transepithelial electrical resistance of greater than 300 Ω·cm⁻² is regarded that a monolayer is dense and intact.

Further, in the step 2), the activity of alkaline phosphatase is detected once every 7 days on average, and a greater ratio of the AP side of the upper chamber to the BL side of a lower chamber indicates a higher degree of cell polarization.

An application of a co-culture model for Caco-2/RAW-264.7 cells induced by lipopolysaccharides in research on absorption of cyanidin-3-glucoside nanoliposomes by the cells is provided.

The present invention builds an in vitro model for cells from an intestinal epithelial cell line Caco-2 located in a top well of the Transwell plate and a macrophage cell line RAW-264.7 located in an outer side well of a substrate, to establish an intestinal inflammatory response for studying a macrophage-mediated absorption mechanism of cyanidin-3-glucoside (C3G) nanoliposomes by the Caco-2 cells, building a data network of “inflammatory response-absorption law-molecular response”, and illustrating an absorption mechanism of cyanidin-3-glucoside in a co-culture system.

### BRIEF DESCRIPTION OF THE DRAWINGS

FIG. 1 is a standard curve of anthocyanin;

FIG. 2 shows morphological structures (TEM pictures) of anthocyanin nanoliposomes under different magnification times;

FIG. 3 is a size distribution diagram of anthocyanin nanoliposomes;

FIG. 4 is a Fourier transform infrared spectrogram of anthocyanin nanoliposomes;

FIG. 5 is a morphological diagram of Caco-2 cells;

FIG. 6 is a change curve of a transepithelial electrical resistance of a monolayer in Caco-2 cells;

FIG. 7 is a standard curve for Lucifer yellow;

FIG. 8 is an effect of cyanidin-3-glucoside (C3G) nanoliposomes on the viability of Caco-2 cells, and compared with a blank group, **P<0.01, and ***P<0.001 (mean+/−SD, n=3);

FIG. 9 is an effect of lipopolysaccharides on the activity of RAW-264.7 cells;

FIG. 10 shows effects of different drugs on expression of inflammatory factors of cells;

FIG. 11 is a relationship between uptake amounts of C3G and C3G nanoliposomes in Caco-2 cells and time (mean+/−SD, n=3);

FIG. 12 is a relationship between transport amounts of C3G NL in Caco-2 cells and concentration, and compared with a C3G group, ***P<0.001, and ****P<0.0001 (mean+/−SD, n=3);

FIG. 13 is a relationship between uptake amounts in Caco-2 cells and temperature, and compared with a blank group (4° C.), ****P<0.0001 (mean+/−SD, n=3); and

FIG. 14 is cell absorption of C3G and C3G nanoliposomes under the effect of endocytosis inhibitors in a co-culture model.

### DESCRIPTION OF THE EMBODIMENTS

The present invention is further described in detail below with reference to the drawings and specific experimental examples of the specification, so as to better understand this technical solution.

Experimental example: Preparation and stability analysis of cyanidin-3-glucoside (C3G) nanoliposomes

Materials and reagents: cyanidin-3-glucoside (purity >95%), cholesterol and lecithin (soybean), sodium dihydrogen phosphate (NaH2PO4), disodium hydrogen phosphate (Na2HPO4), distilled water, trichloromethane, and ethyl ether.

### Experimental Methods

Determination of an optical density of anthocyanin and drawing of a standard curve of the optical density: 2 mg of cyanidin-3-glucoside was accurately weighed and transported to a volumetric flask, phosphate buffered saline (PBS, pH =6.8) was added to dilute it to a graduated line of the volumetric flask, and anthocyanin was fully dissolved by shaking well to obtain an anthocyanin stock solution. 1 mL of the stock solution was diluted with PBS by different concentrations (0 mg/mL, 0.01 mg/mL, 0.02 mg/mL, 0.03 mg/mL, 0.04 mg/mL, and 0.05 mg/mL), an optical density was determined at a wavelength of 531 nm with the PBS as a blank control, and a standard curve was drawn with the concentration as an abscissa and the optical density as an ordinate.

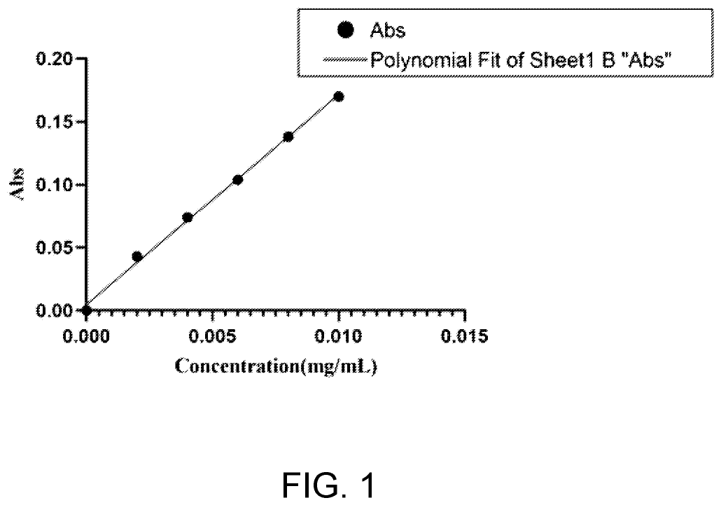

Results showed that the concentration and optical density of the anthocyanin had a good linear relationship in a range from 0.002 mg/mL to 0.01 mg/mL, and the standard curve Y of the optical density of the anthocyanin was equal to 16.643X+0.005, where R²=0.9971. The standard curve obtained was as shown in FIG. 1.

Preparation of anthocyanin nanoliposomes: anthocyanin nanoliposomes were prepared with a reverse-phase evaporation method, 90 mg of soybean lecithin and 30 mg of cholesterol (3 : 1) were accurately weighed in a black cap bottle, and then were dissolved in 18 mL of an organic solvent (chloroform : ethyl ether =10 mL : 8 mL). A sample solution was prepared and 2 mg of anthocyanin was dissolved in 10 mL of PBS (pH 6.8). 1 mL of the sample solution was sucked and added to the above organic solvent. A mixed solution was added to a magnetic rotor and stirred on a magnetic stirrer for 40 min, and ultrasonic treatment was performed for 10 min to form a stable milky white (water in oil) solution. The milky solution was transferred into a round-bottom flask, the flask was uniformly rotated at 41° C. and 45 rpm for evaporation to remove the organic solvent, and when a uniform lipid membrane was formed at the bottom of the flask, 40 mL of PBS was added to ultrasonically elute the membrane. Ultrasonic treatment was performed in an ultrasonic water bath for 10-15 min to obtain an anthocyanin liposome suspension, and the suspension was stored in a refrigerator for later use.

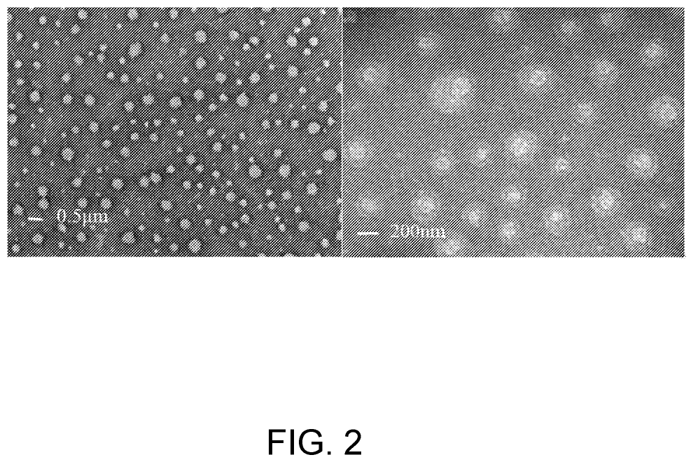

Through a preparation process for anthocyanin based on response surface methodology, a size of the finally prepared anthocyanin nanoliposomes was mostly in a range from 90 nm to 250 nm, and an encapsulation rate thereof was 67.35%+/−1.27%. Results of the anthocyanin nanoliposomes observed by a transmission electron microscope were as shown in FIG. 2. The results showed that the anthocyanin nanoliposomes were distributed in oval or spherical shape and had a vesicular structure. Particles were dispersed independently from each other and had a more obvious hollow structure, and a size thereof was about 200 nm, which was basically consistent with a result determined by a particle-size meter. Meanwhile, the results showed that size distribution of the anthocyanin nanoliposomes was not uniform. Relevant researches showed that the size might be reduced and its distribution was more uniform by some mechanical methods, such as ultrasonic treatment, homogenization, and extrusion. In subsequent tests, some methods (homogenization and ultrasonic treatment) were needed to reduce the size and make its distribution more uniform.

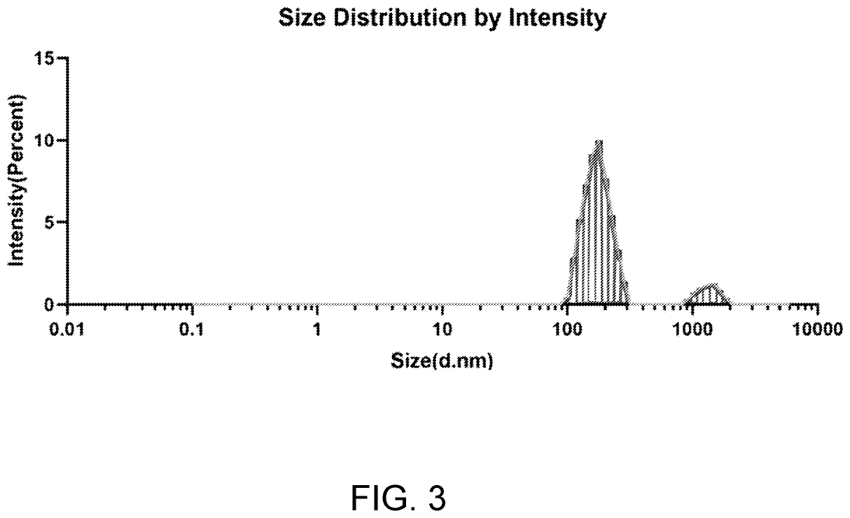

Determination of a size of liposomes: a size of liposomes was measured with a Malvern particle size analyzer. First, 1 mL of a liposome suspension was sucked, a proper amount of water was added for dilution, then a diluted suspension was sucked into a special detection container, and parameters of the analyzer were set (a refractive index thereof was set to be 1.33, that is a refractive index of water, and a buffer system was ultra-pure water). A sample to be tested was repeatedly detected for three times, and size results and size distribution of the sample were recorded, as shown in FIG. 3.

Determination of an encapsulation rate of liposomes: after the preparation of liposomes, an encapsulation rate needed to be determined. The determination of the encapsulation rate was mainly to verify an encapsulation effect of anthocyanin. A detection method for an encapsulation rate of liposomes was as follows: first, 1 mL of prepared anthocyanin liposomes and blank liposomes were sucked into a 10 mL volumetric flask, respectively, centrifugation was performed at 8,000 rpm for 30 min after a constant volume was reached, a supernatant was taken, and an optical density was detected at 531 nm by using the blank liposomes as a control, where a detected result was a concentration (C1) of free anthocyanin after the optical density was substituted into a standard curve equation. Then, 1 mL of prepared anthocyanin liposomes and blank liposomes were sucked into a 10 mL volumetric flask, respectively, 4.5 mL of absolute ethanol was added, centrifugation was performed under the same conditions after a constant volume was reached, a supernatant was taken, and the optical density was detected and substituted into an equation to obtain a total anthocyanin concentration (C2). A calculation formula for an encapsulation rate and a calculation formula for a leakage rate were (1-1) and (1-2) as follows:

$$Encapsulation rate ( \% ) = \frac{C 2 - C 1}{C 2} \times 1 \; 00 \% \qquad ( 1 - 1 )$$

$$Leakage rate ( \% ) = ( 1 - \frac{E E t}{E E 0} ) \times 1 \; 00 \% \qquad ( 1 - 2 )$$

where EEₜ represented an encapsulation rate of anthocyanin in nanoliposomes at a time t, and EE₀ represented an encapsulation rate of anthocyanin in nanoliposomes during initial preparation.

Representation of anthocyanin liposomes: an internal submicroscopic structure of nanoliposomes was observed and determined with a transmission electron microscope. The prepared anthocyanin liposomes were diluted with distilled water for a specific number of times, then ultrasonic dispersion was performed for about 5 min, a small amount of sample was sucked with a pipette after the ultrasonic dispersion and was dripped onto a special carrier net for the transmission electron microscope, phosphotungstic acid was added for negative staining, and after the surface was dry, observation was performed.

Infrared analysis of anthocyanin liposomes: a composition and structure of anthocyanin nanoliposomes were represented and identified with a Fourier transform infrared spectrum. The prepared anthocyanin nanoliposomes and blank liposomes were freeze-dried, 1.5 mg of dried anthocyanin nanoliposomes and blank liposomes were taken, respectively, 200 mg of dried potassium bromide powder was added and was mixed and ground tin an agate mortar, finely ground powder was added to a pressing membrane, and pressing was performed, to obtain a specific uniform sample sheet. Then the sample sheet was put into a sample cell for determination.

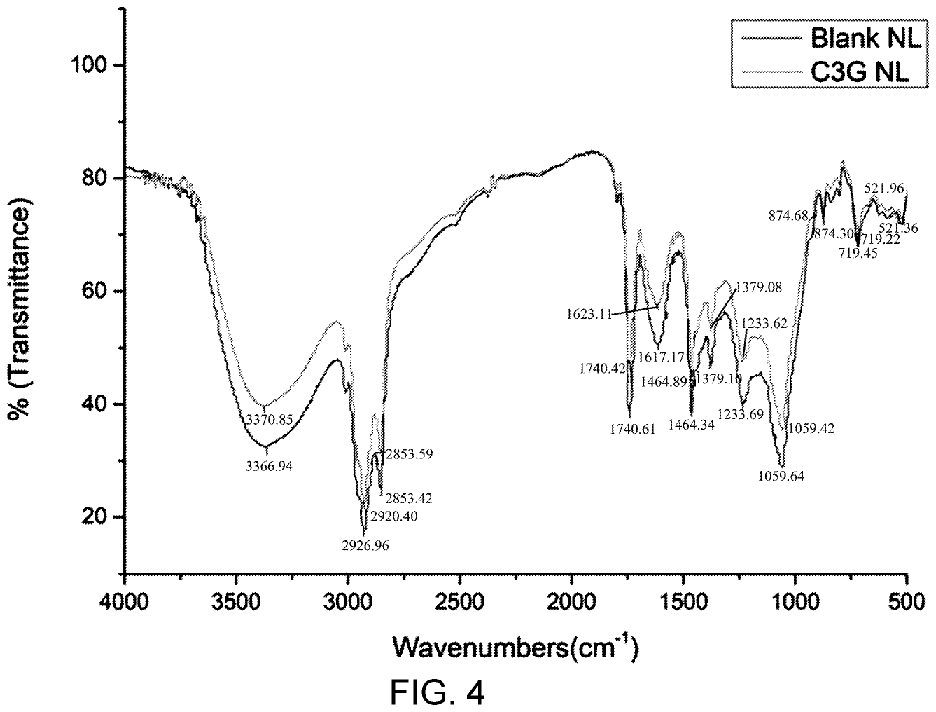

Through analysis of the Fourier transform infrared spectrum, as shown in FIG. 4, it might be found that a structure of liposome-embedded anthocyanin remained unchanged before embedding, and a characteristic absorption peak of groups in a functional group region was still retained, which indicated that the liposome-embedded anthocyanin was not damaged.

### Research on Cellular Absorption of Anthocyanin Nanoliposomes

Materials and reagents: 10 mmol/L of β-CD, 10 μg/mL of chlorpromazine (0.1 ml), 2 μmol/L of cytochalasin D, a 15 ml centrifuge tube, a 50 mL centrifuge tube, a 5 mL pipette tip, a cell cryopreservation tube, a breathable 25 cm² cell culture flask, an airtight 25 cm² cell culture flask, a DMEM, PBS, a trypsin-EDTA solution, cryopreservation grade DMSO, cell grade DMSO, a 6-well plate, a 12-well plate, a 24-well plate, a 48-well plate, and a 96-well plate. In cell culture, double antibody, a disposable sterile syringe, a sterile filter membrane, L-glutamine, a HEPES buffer solution, a nonessential amino acid solution, fetal bovine serum, distilled water, absolute ethanol, CO₂, and liquid nitrogen were used.

### Experimental Methods

Cell culture: Caco-2 cells were cultured in a MEM medium containing 20% of fetal bovine serum, 1% of HEPES, 1% of NEAA, 1% of L-glutamine, 1% of sodium pyruvate, 100 U/mL of penicillin, and 100 mg/mL of streptomycin; and RAW-264.7 cells were cultured in a DMEM medium containing 10% of fetal bovine serum, 1% of HEPES, 1% of NEAA, 1% of L-glutamine, 100 U/mL of penicillin, and 100 mg/mL of streptomycin. Culture conditions included pH 7.4, 5% CO₂, 37° C., 95% air, and constant humidity. The media were changed once every 1-2 days. All experiments were carried out 21 days after inoculation. At this time, the monolayer intactness of the Caco-2 cells should exceed 300 cm² TEER/Ω.

Cell co-culture: Caco-2 cells were inoculated onto a 6-well Transwell plate at a density of 1×10⁵ cells/well. A culture medium was changed once every 1-2 days in 21 consecutive days. RAW-264.7 cells were inoculated onto a side wall of a substrate at a density of 1×10⁵ cells/well, and were cultured in 5% carbon dioxide at 37° C. for 48 h, to promote obvious adhesion of the cells to the side wall of the substrate. After 48 h, the culture medium was replaced with a serum-free DMEM. It was intended to evaluate the absorption of C3G liposomes in a co-culture model.

Cytotoxicity tests: according to manufacturer's instructions, cell viability was determined with a CCK-8 (Nanjing Jiancheng Biological Engineering Research Institute, Nanjing, China). In short, Caco-2 cells were treated with C3G liposomes at concentrations of 0.1 mg/mL, 0.2 mg/mL, 0.3 mg/mL, and 0.4 mg/mL, incubated for 24 h, then washed with PBS once, and added with a CCK-8 solution. Incubation was performed at 37° C. for 4 h, and an optical density at 450 nm was determined. RAW-264.7 cells were treated with LPS at concentrations of 0.5 μg/mL, 1 μg/mL, 1.5 μg/mL, 2μg/mL, and 2.5 μg/mL, incubated for 24 h, then washed with PBS once, and added with a CCK-8 solution. Incubation was performed at 37° C. for 4 h, and an optical density at 450 nm was determined. CCK-8 detection was performed with the above method, and results thereof were results relative to control cells treated with media only.

Measurement of a transepithelial electrical resistance: a transepithelial electrical resistance of cells was measured once every three days on average. Before measurement of the transepithelial electrical resistance, an electrode was immersed in 70% ethanol to be disinfected for 15-20 min, and then put into an HBSS for 15 min of balance after the natural environment was dry. Then two ends of the electrode were vertically inserted into upper and lower pools in each well of a culture chamber of a 24-well Transwell in sequence to measure the resistance. It was noted that the ends of the electrode could not be in contact with a bottom of the chamber. Measurement was performed for three times at any point in each well, results (Rₜ) were recorded, and a resistance (Ro) of a blank well was measured; and according to a calculation formula for a transepithelial electrical resistance TEER=(Rₜ−R₀)×S (S was an effective membrane area), the transepithelial electrical resistance was calculated. Generally, the transepithelial electrical resistance of greater than 500 Ω·cm⁻² was regarded that a monolayer was dense and intact. A larger TEER indicated denser monolayers and was generally not greater than 1,000 Ω·cm⁻².

### Penetrant Inspection for Lucifer Yellow

Drawing of a standard curve for Lucifer yellow: 0.005 μg/mL, 0.01 μg/mL, 0.025 μg/mL, 0.05 μg/mL, 0.1 μg/mL, and 0.25 μg/mL of standard solutions for Lucifer yellow were prepared, the fluorescence intensities (an excitation wavelength: 427 nm, and an emission wavelength: 536 nm) of the standard solutions for Lucifer yellow were measured with a fluorescence spectrophotometer, and linear regression was performed with the fluorescence intensity as an ordinate and the concentration (m/mL) as an abscissa. A standard curve was drawn.

Transport tests for Lucifer yellow: Caco-2 cells grew to 21 days in a Transwell, and then a culture medium was sucked and discarded. A preheated HBSS was added to both an AP side and a BL side for cleaning twice and was sucked and discarded, and then a preheated HBSS was added to perform culture under the conditions of 37° C. and 5% CO₂ for 20 min. An HB SS was sucked and discarded, 0.5 mL of Lucifer yellow CH (μg/mL) was added on an A side, 1.5 mL of the HBSS was added on a B side, culture was performed under the conditions of 37° C., 5% CO₂, and 90% saturated humidity for 2 h, 100 ul of a sample was taken from a BL pool at 60 min and 120 min, and the same volume of a blank HB SS was replenished. Under the condition of Ex=427 nm and Em=536 nm, the fluorescence intensity was measured according to a standard curve and a transport formula. A concentration and permeability coefficient of a transport solution for the Lucifer yellow CH on the BL side were calculated.

$${P}_{app} = \frac{d Q}{d t} \times \frac{1}{A \times C o}$$

where Papp was an apparent permeability coefficient, in cm/s, and dQ/dt was the transmittance of Lucifer yellow in unit time, in μg/s; A was a surface area of a membrane, in cm²; and C₀ was an initial concentration of the Lucifer yellow on the AP side, in μg/mL.

Determination of the activity of alkaline phosphatase: the activity of alkaline phosphatase was determined with an AKP kit. When Caco-2 cells grew to 7 days, 14 days, and 21 days in a 24-well transwell, cell monolayers were gently washed with a Hank's balanced salt solution twice, the cells were incubated at 37° C. for 20 min, and the solution inside and outside wells was sucked. The activity of alkaline phosphatase on AP and BL sides in the cell monolayers was determined by a kit method.

Determination of the content of inflammatory factors in a co-culture model: levels of inflammatory factors IL-6, IL-8, IL-1β, and TNF-α in a supernatant of Caco-2 cells in an LPS-induced co-culture model were determined with an ELISA kit.

Effect of time on uptake in a co-culture model: 0.2 mg/mL of a C3G and C3G NL solution was prepared with a DMEM, 0.1 ml of a sample solution was added on an AP side, 0.6 mL of a DMEM was added on an BL side, culture was performed in an incubator at 37° C. for 1 h, 2 h, 3 h, 4 h, 5 h, and 6 h, three wells were provided at each time point, a drug-containing buffer solution was discarded after incubation, precooled PBS was added to stop cellular uptake, and cell monolayers were quickly washed for three times. 200 μL of a cell lysis solution was added to each well, lysis was performed on ice for 5 min, cells were scraped into an Ep tube with a cell scraper, and the cells were ultrasonically disrupted. A cell suspension was dissolved with methanol and centrifuged at 15,000 rpm for 20 min, then a supernatant was taken, and the content of C3G was determined with HPLC.

Effect of concentration on uptake in a co-culture model: 0.02 mg/ml, 0.04 mg/ml, mg/ml, 0.08 mg/ml, and 0.1 mg/ml of C3G and C3G NL solutions were prepared, transport from an AP side to a BL side and transport from the BL side to the AP side were evaluated, 0.1 ml of a sample was added on the AP side, 0.6 ml of a sample was added on the BL side, three wells were provided, culture was performed in an incubator for 2 h, a culture medium was discarded after culture, and uptake was stopped using an HBSS. The content of the sample on the BL side was determined with HPLC.

Effect of temperature on uptake in a co-culture model: 0.2 mg/mL of a C3G and C3G NL solution was prepared with an HBSS, 0.1 ml of a sample solution was added on an AP side, 0.6 mL of an HBSS was added on an BL side, cells were cultured in environments at 37° C. and 4° C. for 2 h, three wells were provided at each temperature, a culture medium was discarded after culture, and uptake was stopped using an HBSS. The content of a sample on the BL side was determined with HPLC.

Effect of an endocytosis inhibitor on uptake in a co-culture model: 0.2 mg/mL of a C3G and C3G NL solution was prepared with an HBSS, 0.1 ml of a sample solution was added on an AP side, 0.6 mL of an HBSS was added on a BL side, an MEM, 10 mmol/L (4 mg/ml) of β-CD,10 μg/mL of chlorpromazine, 2 μmol/L (1 μg/ml) of cytochalasin D (0.1 ml) were added and sucked out after 30 min, cells were cultured in an environment at 37° C. for 1 h, three wells were provided, a culture medium was discarded after culture, and uptake was stopped using an HBSS. The content of a sample was determined with HPLC, and Papp was calculated.

Uptake of coumarin-6 tracing nanoliposomes in a co-culture model: coumarin nanoliposomes were prepared, cells were inoculated in a 96-well plate by density, after 4 days of culture, the cells were washed with PBS twice and cultured with PBS for 20 min, the PBS was discarded, PBS containing C6-NL was added for culture, effects of different time and temperatures on cellular uptake of C6-NL were inspected, and uptake was stopped using PBS after culture. A nuclear staining agent DAPI was added to perform incubation for 15 min, the cells were washed with PBS for three times, and distribution of C6-NL in the cells was observed under a fluorescence microscope.

### Results and Discussion

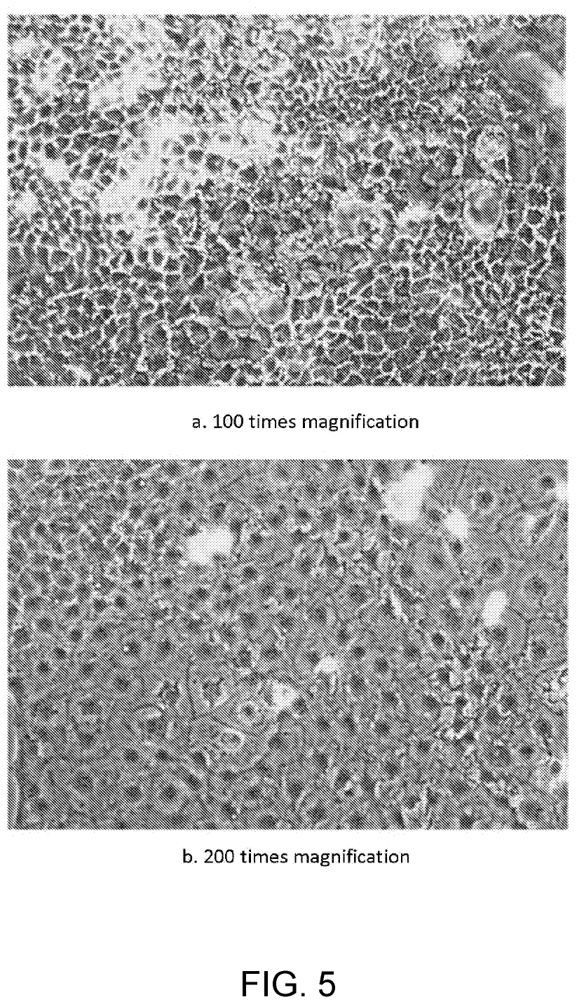

Morphological observation of Caco-2 cells: Caco-2 cells were adherent cells. Under an inverted microscope, the wells were in polygonal and flat shape and were arranged neatly. At 100 times magnification, the cells were distributed individually, had clear edges, and grew well. At 200 times magnification, it might be observed that the cells after fusion are closely connected and in the shape of “a paving stone”, as shown in FIG. 5.

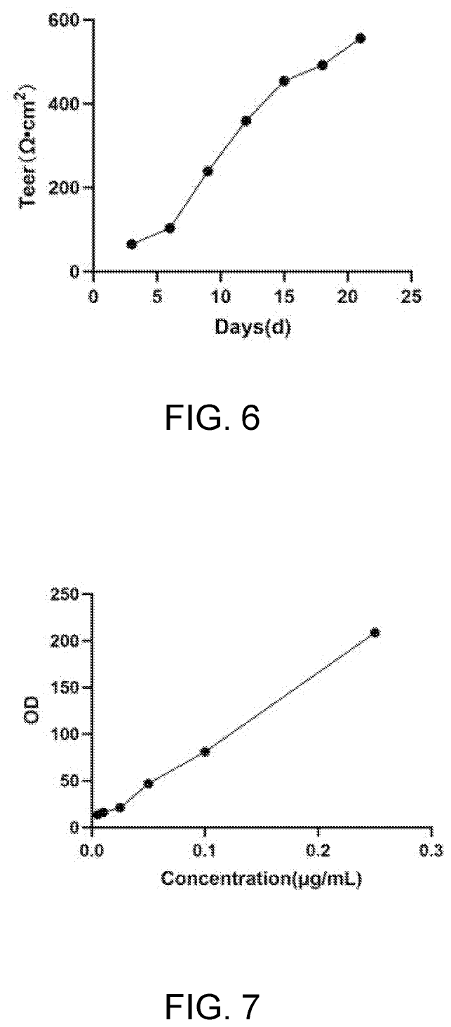

Determination of monolayer intactness of Caco-2 cells: an electrical resistance of a cell model reflected the density and intactness of cells to some extent. Generally, the TEER was between 50 Ω·cm² and 600 Ω·cm². A larger electrical resistance indicated denser monolayers. After the model was built successfully, a transport experiment might be carried out. The electrical resistance measured by a resistance meter was shown in FIG. 6. It was seen from FIG. 6 that the density of the Caco-2 cell model tended to be increasing in 21 days. The electrical resistance reached 556 Ω·cm² on the 21st day.

The electrical resistance increased slowly in the previous week, and was about 100 Ω·cm². It might be because the cells had weaker activity and were divided slowly after being digested by trypsin. The electrical resistance began to increase rapidly in the second week. At this time, the cells were divided rapidly and differentiated into a special structure of intestinal epithelial cells. The electrical resistance increased slowly in the last week. Cell differentiation was basically completed, and cell membranes became denser.

Determination of the permeability of Lucifer yellow: a standard curve equation for Lucifer yellow was obtained, that is, y=803.6x+5.623, where R²=0.9974, and a linear range was 0.005-0.25 μg/mL; and a standard curve diagram was as shown in FIG. 7. 20 μg/mL of Lucifer yellow was added to Caco-2 cells and sampled on a BL side at 60 min and 120 min. Under the condition of Ex=427 nm and Em=536 nm, the fluorescence intensities were detected to be 26.512 +/−1.502 and 27.035 +/−3.184 (n=3), respectively. According to a formula, Papp was calculated to be 3.03×10⁻⁸ cm/s, which was less than 5×10⁻⁷ cm/s. It indicated that Caco-2 cells had formed intact monolayers after being cultured in a Transwell plate for 21 days, and might be used for a transport experiment.

Research on cell polarity: it was reported in literatures that when an ALP activity ratio (AP/BL) on two sides of a cell monolayer was greater than 3, cell differentiation was asymmetric, resulting in polarity differentiation. It might be seen from Table 1 that ALP activity ratios (AP/BL) were 2.301+/−0.41, 3.727 +/−0.44, and 8.662 +/−0.52 on the 7th, 14th, and 21st day, respectively, so that the ratio increased with time; and the ALP activity ratio (AP/BL) on the 21st day was about four times the activity ratio on the 7th day, which indicated that ALP was significantly polarized, and a structure of monolayers of Caco-2 cells had a characteristic of polarity.

TABLE 1
Relationship between a ratio of alkaline phosphatase
activities on two sides of a monolayer of a Caco-2
cell and time (mean +/− SD, n = 3)

| T/d [symbol] | 7 d [symbol] | 14 d [symbol] | 21 d [symbol] |
|---|---|---|---|
| ALP/(AP/BL) [symbol] | 2.301 ± 0.41 [symbol] | 3.727 ± 0.44 [symbol] | 8.662 ± 0.52 [symbol] |

In conclusion, after 21 days of culture, morphological characteristics of Caco-2 cells were similar to those of small intestinal epithelial cells, cell membranes were dense and intact, and polarization on two sides of a membrane was obvious. Therefore, the built Caco-2 cell model was consistent with requirements of a drug transport experiment, and might serve as an in vitro model to inspect small intestinal absorption of drugs.

### Toxicity Tests

Effect of C3G nanoliposomes on the viability of Caco-2 cells: effects of C3G NL and C3G on the viability of Caco-2 cells were inspected with a CCK-8 method. The Caco-2 cells were inoculated onto a 96-well plate having each well of 100 μL at a density of 1×10⁵ cells/mL, and 200 μL of PBS was added to a peripheral well of the 96-well plate, to provide a humidity environment. Culture was performed in an incubator at 37° C., and after the Caco-2 cells adhered to a wall, 100 μL of a drug-containing DMEM was added, to cause final concentrations of C3G NL and C3G to be 0 mg/mL, 0.1 mg/mL, 0.2 mg/mL, 0.3 mg/mL, and 0.4 mg/mL. The cells were divided into groups and subjected to corresponding drug intervention, each group was provided with six wells, and a control group was provided with six wells (with cells, without drugs, and added with a DMEM medium). After incubation for 24 h, 20 μL of a CCK-8 solution was added to each well, incubation was performed in an incubator at 37° C. for 1 h, and then an optical density (OD) at a wavelength of 490 nm was detected with a microplate reader and was calculated according to the following formula:

Cell viability (%)=OD_{treatment well}/OD_{control well}×100%

where the OD_{treatment well} represented an optical density of cells after addition of drugs, and the OD_{control well} represented an optical density of cells in a blank control.

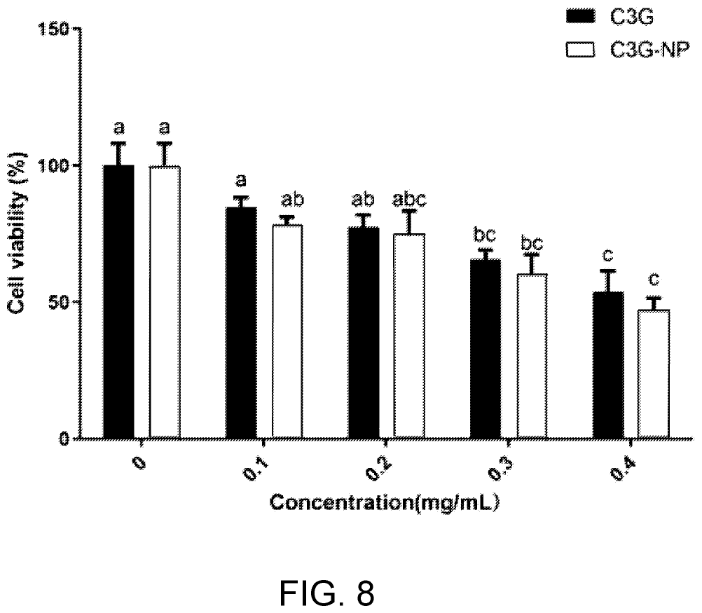

It might be seen from FIG. 8 that after the cells were incubated with a series of concentrations of C3G NL and C3G for 24 h, the viabilities thereof were greater than 70% when the concentrations of C3G NL and C3G were less than 0.2 mg/mL. Compared with a blank group, when the concentrations of C3G NL and C3G were 0.2 mg/mL and below, the cell viability did not change significantly (P>0.05), and when the concentrations of BA and BA SLN were 0.3 mg/mL and 0.4 mg/mL, the cell viability decreased significantly (P<0.01, P<0.001), which indicated that the C3G NL and C3G did not cause damage to the cells at the concentration of 0.2 mg/mL.

### Effect of Lipopolysaccharides on the Viability of RAW-264.7 Cells:

An effect of LPS on the viability of RAW-264.7 cells was inspected with a CCK-8 method. The RAW-264.7 cells were inoculated onto a 96-well plate having each well of 100 μL at a density of 1×10⁵ cells/mL, and 200 μL of PBS was added to a peripheral well of the 96-well plate, to provide a humidity environment. Culture was performed in an incubator at 37° C., and after the RAW-264.7 cells adhered to a wall, 100 μL of a drug-containing DMEM was added, to cause final concentrations of LPS to be 0 μg/mL, 0.5 μg/mL, 1 μg/mL, 1.5 μg/mL, 2μg/mL, and 2.5 μg/mL. The cells were divided into groups and subjected to corresponding drug intervention, each group was provided with six wells, and a control group was provided with six wells (with cells, without drugs, and added with a DMEM medium). After incubation for 24 h, 20 μL of a CCK-8 solution was added to each well, incubation was performed in an incubator at 37° C. for 1 h, and then an optical density (OD) at a wavelength of 490 nm was detected with a microplate reader and was calculated according to the following formula:

Cell viability (%)=OD_{treatment well}/OD_{control well}×100%

where the OD_{treatment well} represented an optical density of cells after addition of drugs, and the OD_{control well} represented an optical density of cells in a blank control.

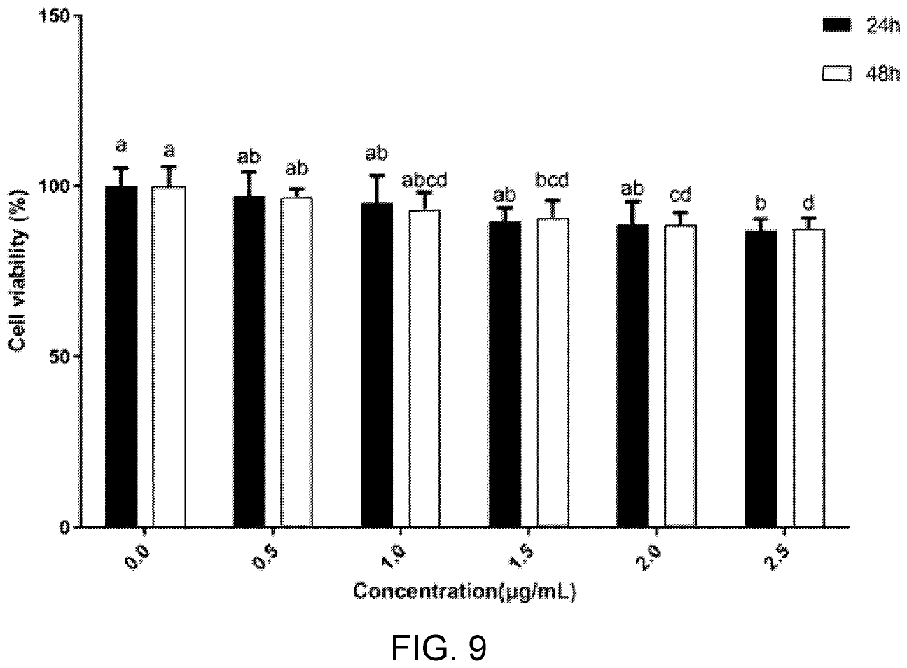

It might be seen from FIG. 9 that after the cells were incubated with a series of concentrations of LPS for 24 h, the viabilities thereof were greater than 70% when the concentration of LPS was within a range of 2.5 μg/mL, which indicated that the LPS did not cause effect to the viability of cells within 2.5 μg/mL.

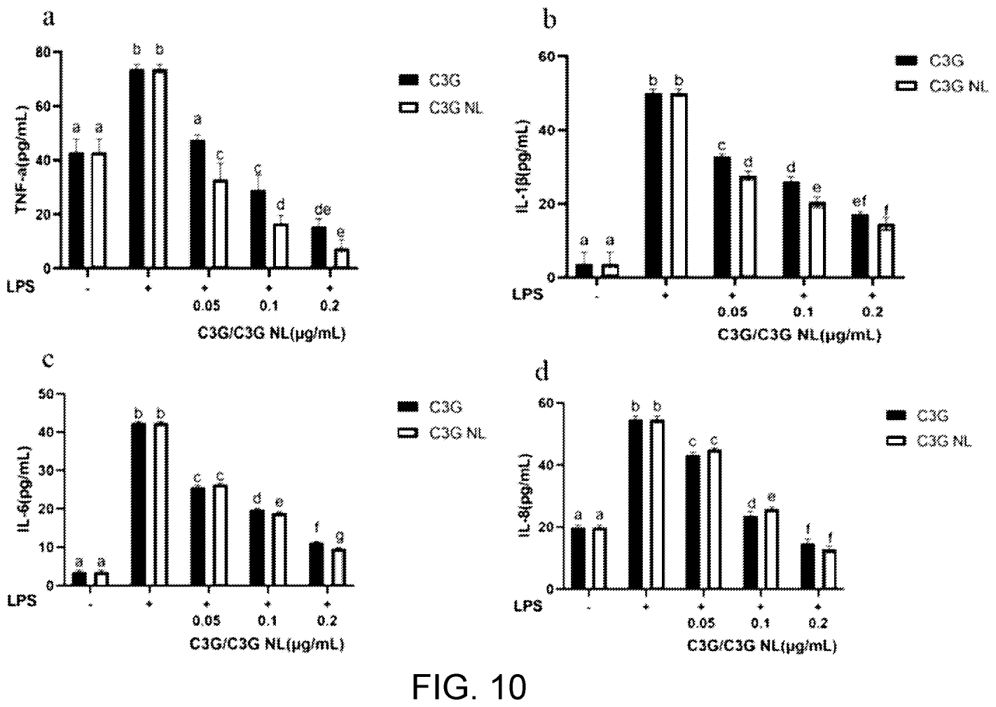

Effect of C3G NL and C3G on an anti-inflammatory action in a co-culture model: TNF-a, IL-1β, IL-6, and IL-8 were important inflammatory factors in the process of inflammation. As shown in FIG. 10, LPS significantly increased the secretion of inflammatory factors (P<0.001) and the production of inflammatory factors was reduced in a dose-dependent manner after anthocyanin and anthocyanin nanoliposome pretreatment. To compare the effects of C3G and C3G liposome groups on the production of inflammatory factors of cells, two compounds with the same concentration were added under the same conditions. As shown in FIG. 10, LPS could significantly promote the production of inflammatory factors, and when the drug concentration was higher, the content of inflammatory factors was lower. As shown in a and b in FIG. 10, the inhibitory effect of C3G liposomes on inflammatory factors was greater than that of C3G with the same concentration. As shown in b and c in FIG. 10, compared with an LPS group, when the drug concentration was higher, the content of IL-1β and IL-6 in a cell supernatant was lower.

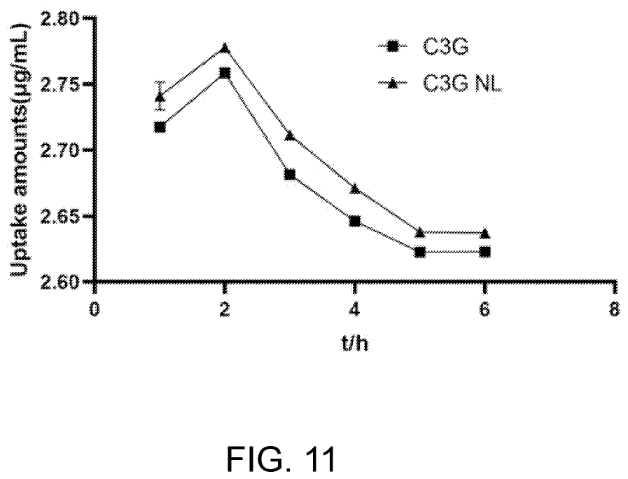

Effect of time on uptake in a co-culture model: it might be seen from FIG.11 that when the drug concentration is 0.2 mg/mL, uptake amounts of C3G and C3G NL in Caco-2 cells increased first and then decreased with time of 0-6 h and reached the maximum at 2 h. Results showed that the maximum time point for the uptake amounts of C3G and C3G NL in Caco-2 cells was 2 h, and 2 h would be a time point for subsequent uptake experiments.

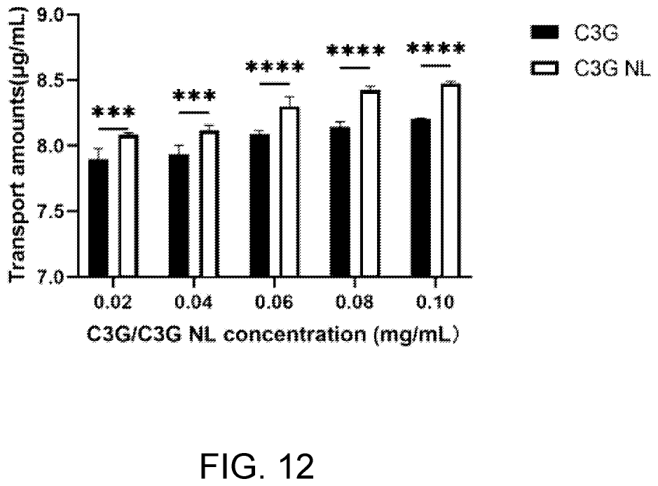

Effect of concentration on transport in a co-culture model: it might be seen from FIG. 12 that at 1 h, transport amounts of C3G and C3G NL in Caco-2 cells increased with the increase of concentration and would not be saturated; and at the same concentration, transport amounts of a C3G NL group were significantly higher than those of a C3G group (P<0.001, P<0.0001). Results showed that carriers might not be needed in the transport of C3G and C3G NL, which belonged to concentration-dependent passive diffusion or endocytosis.

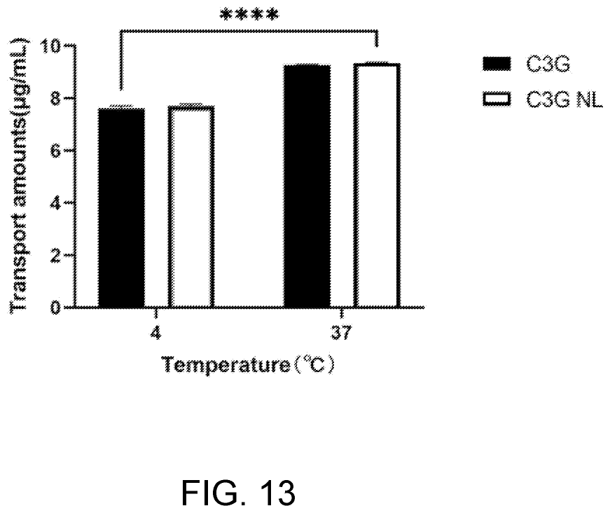

Effect of temperature on transport in a co-culture model: it might be seen from FIG. 13 that uptake amounts of C3G and C3G NL in Caco-2 cells increased significantly with the increase of temperature (4° C., 37° C.) (P<0.0001). Results showed that the uptake of C3G and C3G NL on cells was energy-dependent, and might be active uptake or endocytosis.

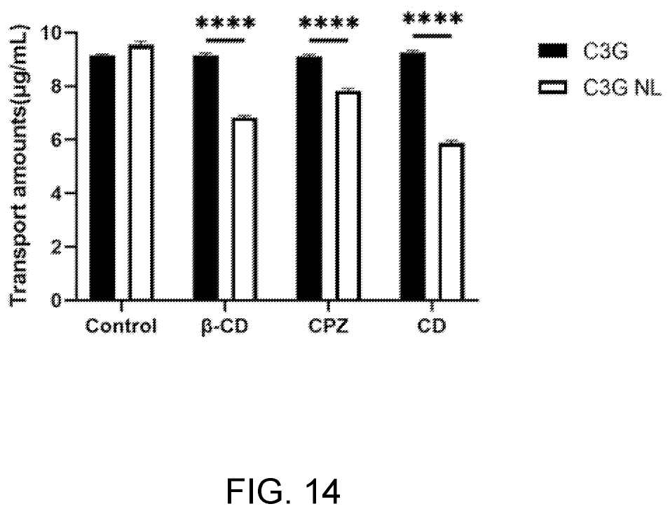

Effect of an endocytosis inhibitor on transport in a co-culture model: As shown in FIG. 14, compared with a blank group, after the addition of an endocytosis inhibitor, that is, water-soluble methyl β cyclodextrin (β-CD), chlorpromazine (CPZ), and cytochalasin D, C3G had no significant changes in transport amounts of the co-culture model (P>0.05); and compared with the blank group, the cellular absorption of C3G liposomes would be inhibited by β-CD. This was because the water-soluble methyl β cyclodextrin (β-CD) might react with cholesterol to a certain extent, and then the cholesterol on a cell membrane would be removed by it, which led to the destruction of a cholesterol-containing domain on the cell membrane; and such domain would participate in caveolae and clathrin-mediated endocytosis. Therefore, the caveolae and clathrin-mediated endocytosis might be effectively inhibited by (3-CD.

Compared with a blank group, cellular uptake of C3G nanoliposomes was inhibited by CPZ. This was because endocytosis of most nanomaterials mainly depended on clathrin-mediated endocytosis, and chlorpromazine (CPZ) might significantly affect clathrin-mediated endocytosis (p<0.05).

Compared with a blank group, CD significantly reduced cellular absorption of anthocyanin nanoliposomes (p<0.05), and the content of anthocyanin in cells was reduced. It showed that macropinocytosis also participated in cellular uptake of anthocyanin nanoliposomes to a certain extent. Because cytochalasin D (CD) might inhibit actin filament-related absorption, and might affect actin-related endocytosis. Actin was also used to study influence factors of a macropinocytosis pathway because it strictly controlled a macropinocytosis inhibitor.

What is claimed is:

1. A method for building a co-culture model for Caco-2/RAW-264.7 cells induced by lipopolysaccharides, wherein the method comprising the following steps:
1) culturing Caco-2 cells in an MEM medium and RAW-264.7 cells in a DMEM medium under culture conditions of pH 7.4, 5% CO₂ , 37° C., 95% air, and constant humidity for 21 days, wherein the media are changed once every 1-2 days;
2) inoculating the Caco-2 cells cultured in the step 1) onto an AP side of an upper chamber of a 24-well Transwell plate having a PC membrane of 4μm at a density of 1×10⁵ cells/well, and incubating under the same culture conditions as those in the step 1) for 21 days, wherein a culture medium is changed once every 24 h;
3) inoculating the RAW-264.7 cells onto a 24-well plate at a density of 1×10⁵ cells/well, and incubating under the same culture conditions as those in the step 1) for 1 day;
4) transferring the upper chamber of the Transwell plate loaded with the Caco-2 cells incubated for 21 days to the 24-well plate loaded with the RAW-264.7 cells incubated for 1 day, wherein at this time, the Caco-2 cells are located on the AP side, and the RAW-264.7 cells are located on a BL side;
5) dissolving lipopolysaccharides in PBS to prepare a 50 μg/mL stock solution, filtering through a 0.22 μm pinhole filter, and diluting the stock solution into a 1 μg/mL LPS-10% DMEM medium with a 10% DMEM medium; and
6) adding the 1 μg/mL LPS-10% DMEM medium on the BL side, incubating for 3 h, detecting the concentration of an inflammatory factor on the AP side with an ELISA kit, and forming an intestinal inflammation model.

2. The method for building the co-culture model for Caco-2/RAW-264.7 cells induced by lipopolysaccharides according to claim 1, wherein in the step 1), the MEM medium contains 20% of fetal bovine serum, 1% of HEPES, 1% of NEAA, 1% of L-glutamine, 1% of sodium pyruvate, 100 U/mL of penicillin, and 100 mg/mL of streptomycin.

3. The method for building the co-culture model for Caco-2/RAW-264.7 cells induced by lipopolysaccharides according to claim 1, wherein in the step 1), the DMEM medium contains 10% of fetal bovine serum, 1% of HEPES, 1% of NEAA, 1% of L-glutamine, 100 U/mL of penicillin, and 100 mg/mL of streptomycin.

4. The method for building the co-culture model for Caco-2/RAW-264.7 cells induced by lipopolysaccharides according to claim 1, wherein in the step 2), a transepithelial electrical resistance of the cells is detected once every three days on average, and the transepithelial electrical resistance of greater than 300 Ω·cm⁻² is regarded that a monolayer is dense and intact.

5. The method for building the co-culture model for Caco-2/RAW-264.7 cells induced by lipopolysaccharides according to claim 1, wherein in the step 2), the activity of alkaline phosphatase is detected once every 7 days on average, and a greater ratio of the AP side of the upper chamber to the BL side of a lower chamber indicates a higher degree of cell polarization.

6. An application of the co-culture model for Caco-2/RAW-264.7 cells induced by lipopolysaccharides according to claim 1 in research on absorption of cyanidin-3-glucoside nanoliposomes by the cells.

7. An application of the co-culture model for Caco-2/RAW-264.7 cells induced by lipopolysaccharides according to claim 2 in research on absorption of cyanidin-3-glucoside nanoliposomes by the cells.

8. An application of the co-culture model for Caco-2/RAW-264.7 cells induced by lipopolysaccharides according to claim 3 in research on absorption of cyanidin-3-glucoside nanoliposomes by the cells.

9. An application of the co-culture model for Caco-2/RAW-264.7 cells induced by lipopolysaccharides according to claim 4 in research on absorption of cyanidin-3-glucoside nanoliposomes by the cells.

10. An application of the co-culture model for Caco-2/RAW-264.7 cells induced by lipopolysaccharides according to claim 5 in research on absorption of cyanidin-3-glucoside nanoliposomes by the cells.
